(19) United States
(12) Patent Application Publication
(10) Pub. No.: US 2004/0137054 A1
(43) Pub. Date: Jul. 15, 2004

(54) Stable pharmaceutical formulation for a combination of a statin and an ace-inhibitors

(76) Inventors: Alexandra Hager, Munchen (DE); Kristian Lund Henriksen, Syborg (DK)

Correspondence Address: James P Zeller, Marshall Gerstein & Borun, Sears Tower Suite 6300, 233 South Wacker Drive, Chicago, IL, 60606-6357, US

(21) Appl. No.: 10/477,674

(22) PCT Filed: Apr. 29, 2003
(86) PCT No.: PCT/EP03/04472

(30) Foreign Application Priority Data
May 3, 2002 (DE) 102 19 949.3
May 16, 2002 (DE) 102 22 326.2

(51) Int. Cl. A61K 31/401; A61K 31/375; A61K 31/366; A61K 9/20; A61K 31/355; A61K 31/225; A61K 31/195

(52) U.S. Cl. 424/465; 514/423; 514/460; 514/458; 514/474; 514/548; 514/566

(57) ABSTRACT

The invention relates to a pharmaceutical formulation containing at least one statin and at least one ACE inhibitor, the at least one statin and the at least one ACE inhibitor being separated by a physiologically acceptable inert material.

The invention relates to a stable pharmaceutical formulation comprising a combination of a statin, such as simvastatin, with an ACE inhibitor, such as ramipril.

Statins impede cholesterol biosynthesis by exerting an inhibitory action on the enzyme 3-hydroxy-3-methylglutaryl coenzyme A reductase. They are therefore used in the treatment of hyperlipidaemia. The statins include atorvastatin, rosuvastatin, fluvastatin, pravastatin, cerivastatin and, especially, simvastatin. The synthesis of simvastatin, (1S,3R,7S,8S,8aS)-1,2,3,7,8,8a-hexahydro-3,7-dimethyl-8-{2-[(4R,6R)-tetrahydro-4-hydroxy-2-oxo-2H-pyran-6-yl]-ethyl}-1-naphthyl 2,2-dimethylbutyrate, is described in, for example, U.S. Pat. No. 4,444,784 and EP 0 033 538 B1. Simvastatin is marketed in the form of a film-coated tablet under the name ZOCOR^{R}.

ACE inhibitors, such as lisinopril, enalapril, quinapril, imidapril, fosinopril, moexipril, perindopril, spirapril, cilazapril, captopril, trandolapril and ramipril act as inhibitors of angiotensin-converting enzyme. They are used in the treatment of essential hypertension and heart failure. The preparation and pharmaceutical use of ramipril, 2-{N-[(S)-1-(ethoxycarbonyl)-3-phenylpropyl]-L-alanyl}-(1S,3S,5S)-2-azabicyclo[3.3.0]octane-3-carboxylic acid, and its salts are described in EP 0 079 022 B1.

A fixed combination of a statin with an ACE inhibitor in one presentation form has the advantage that patient compliance is greater than in the case of separate administration. It is known that the lower the number of preparations per administration time-point, the greater the reliability of administration. A fixed combination accordingly improves the safety and reliability of a therapy.

EP 0 457 514 B1 describes the use of ACE inhibitors in combination with a cholesterol-lowering agent, such as pravastatin, in the prophylaxis and treatment of atherosclerosis.

EP 0 459 453 A2 discloses the use of pravastatin, on its own or in combination with an ACE inhibitor, in the prevention or treatment of the risk of restenosis after an angioplasty.

EP 0 461 548 A2 and EP 0 738 512 A1 describe the use of HMG-CoA reductase inhibitors, on their own or in combination with an ACE inhibitor, in the prevention of a second heart attack.

EP 0 482 498 A2 relates to the use of cholesterol-lowering agents, such as pravastatin, on their own or in combination with ACE inhibitors, in the prevention and treatment of diabetes.

U.S. Pat. No. 5,298,497 describes the use of cholesterol-lowering agents, such as pravastatin, on their own or in combination with ACE inhibitors, in reducing the risk of high blood pressure in patients with insulin resistance.

WO 99/11260 relates to the combination of atorvastatin with blood-pressure-lowering agents, inter alia ACE inhibitors.

WO 00/45818 discloses the treatment of diabetic neuropathy with statins, optionally in combination with ACE inhibitors.

WO 01/15674 describes the use of, for example, HMG-CoA reductase inhibitors in combination with ACE inhibitors in the prevention or treatment of certain diseases.

WO 01/76573 relates to the use of, for example, ACE inhibitors together with cholesterol-lowering agents in the prevention of cardiovascular diseases.

It has now been found that, for a fixed statin/ACE inhibitor combination in an oral presentation form, the stability of the active ingredients in the tablet or capsule does not meet requirements. The ACE inhibitors are compounds that have a tendency to decomposition reactions (cf. EP 0 280 999 B1), which are promoted by acids and bases, whereas acid and basic substances are used for stabilisation of the sensitive statins. Accordingly, in the presence of a stabilised statin, the ACE inhibitor decomposes to such an extent that, even after a short storage period, the content of decomposition products is so high that the permissible limit of degradation products is far exceeded.

The problem of the present invention is to provide a pharmaceutical formulation comprising a statin in combination with an ACE inhibitor, the stability of which with respect to degradation of the active ingredients, meets statutory requirements. The active ingredient content should remain the same over a relatively long period and must be subject to virtually no decomposition processes.

The problem underlying the invention is now solved by a pharmaceutical formulation having a content of at least one statin and at least one ACE inhibitor, wherein the at least one statin and the at least one ACE inhibitor are separated by a physiologically acceptable inert material.

In the pharmaceutical formulation according to the invention, the at least one statin may be present with at least one statin stabiliser, and the at least one ACE inhibitor may be present with at least one optional ACE inhibitor stabiliser.

The pharmaceutical formulation according to the invention may furthermore be present with a buffer substance, antioxidant and/or chelate-former as statin stabiliser.

The pharmaceutical formulation according to the invention may furthermore be present with vitamin E, butylated hydroxytoluene, butylated hydroxyanisole, ascorbic acid, ascorbyl palmitate and/or sodium hydrogen sulfite as antioxidant.

The pharmaceutical formulation according to the invention may furthermore be present with disodium ethylenediaminetetraacetic acid, potassium citrate, sodium citrate and/or citric acid as chelate-former.

The pharmaceutical formulation according to the invention may furthermore be present with a buffer substance as ACE inhibitor stabiliser.

The pharmaceutical formulation according to the invention may furthermore be present with sodium dihydrogen phosphate, sodium citrate, sodium carbonate, sodium hydrogen carbonate and/or tris(hydroxymethyl)aminomethane as buffer substance.

The pharmaceutical formulation according to the invention may furthermore be present with a natural polymer, a synthetic polymer, a non-simple sugar and/or an inorganic material as inert material.

In the pharmaceutical formulation according to the invention, the separating physiologically acceptable material may be provided in the form of an encapsulation of the combination of at least one statin and at least one statin stabiliser.

In the pharmaceutical formulation according to the invention, the at least one statin may furthermore be present in admixture with at least one statin stabiliser in the form of particles, especially granules or pellets, it being possible for the particles to be encapsulated by the separating physiologically acceptable material.

The pharmaceutical formulation according to the invention may furthermore be obtainable by encapsulating particles of a mixture of at least one statin and at least one statin stabiliser by the separating physiologically acceptable material.

The pharmaceutical formulation according to the invention may furthermore be obtainable by embedding, and thereby encapsulating, the at least one statin and the at least one statin stabiliser in the separating physiologically acceptable material.

In the pharmaceutical formulation according to the invention, the at least one statin and the at least one statin stabiliser may furthermore be encapsulated by sucrose, metodextrin, gum arabic, wax, polyvinylpyrrolidone, gelatin, cellulose, a cellulose derivative and/or shellac as inert material.

For the pharmaceutical formulation according to the invention, hydroxypropyl methylcellulose, hydroxypropyl cellulose, cellulose acetate and/or ethyl cellulose may be used as cellulose derivative.

In the pharmaceutical formulation according to the invention, the at least one statin and the at least one statin stabiliser may be encapsulated by a polyacrylate, polymethacrylate, polyether and/or polyether alcohol, especially polyethylene glycol, as polymer.

The pharmaceutical formulation according to the invention may be filled into capsules.

The pharmaceutical formulation according to the invention may also be further processed into tablets.

The pharmaceutical formulation according to the invention may be provided in the form of tablets wherein the at least one statin and the at least one ACE inhibitor are separated by a layer or a membrane of a physiologically acceptable inert material of neutral pH.

For a pharmaceutical formulation according to the invention of that kind, calcium phosphate may be provided as inorganic inert material.

For a pharmaceutical formulation according to the invention of that kind, microcrystalline cellulose and/or pregelatinised starch may also be provided as inert material.

A pharmaceutical formulation according to the invention may be provided with atorvastatin, rosuvastatin, fluvastatin, pravastatin, cerivastatin and/or simvastatin as statin.

In the pharmaceutical formulation according to the invention, the at least one statin may in each case be present in the form of the carboxylic acid, a carboxylic acid salt, a lactone, an ester, a hydrate, an alcoholate and/or a tautomer.

A pharmaceutical formulation according to the invention may be present with captopril, ceranapril, zofenopril, lisinopril, enalapril, quinapril, imidapril, fosinopril, moexipril, perindopril, spirapril, cilazapril, trandolapril and/or ramipril as ACE inhibitor.

In the pharmaceutical formulation according to the invention, the at least one ACE inhibitor may in each case be present in the form of the carboxylic acid, an ester, a salt, an acid addition salt, a hydrate and/or a solvate as ACE inhibitor.

It has now been found, surprisingly, that, as a result of encapsulating the stabilised statin in a protective layer, a combination with an ACE inhibitor is capable of forming a stable pharmaceutical formulation. The protective layer therein must be inert with respect to the statin stabilisers and optional ACE inhibitor stabilisers.

A stable pharmaceutical formulation may also be achieved by separation of statin and ACE inhibitor by means of a membrane.

In general, it may be said that the problem underlying the invention is solved by the two active ingredients—statin and ACE inhibitor—being present in separated form in a pharmaceutical formulation.

The active ingredient combination according to the invention may comprise one or more statins, for example atorvastatin, rosuvastatin, fluvastatin, pravastatin, cerivastatin and/or simvastatin. The statins may be used in the form of a lactone, ester (e.g. (C1-C4)alkyl ester), carboxylic acid or carboxylic acid salt, hydrates, alcoholates, tautomers or the like.

As preferred examples of statin salts there may be mentioned: alkali metal salts such as sodium or potassium salts and also ammonium salts that are derived from ammonia or organic amines, such as ethylenediamine, ethylamine, diethylamine, dipropylamine, diisopropylamine, tripropylamine, trihexylamine, tridodedecylamine, dicyclohexylamine, ethanolamine, diethanolamine, triethanolamine, triisopropanolamine, arginine or lysine, and also alkaline earth metal salts, such as magnesium or calcium salts.

Simvastatin is preferably used in the form of a lactone.

Statin degradation processes may be promoted by temperature, moisture, low pH, light and oxygen; cf., for example, WO 00/35 425. Acid sensitive statins, such as atorvastatin, rosuvastatin, pravastatin, fluvastatin and cerivastatin are therefore formulated in the highly alkaline range at pH 9 to 10. In those cases, buffers act as stabilisers; cf., for example, WO 00/35 425. Adequate simvastatin stability can be achieved by means of stabilisers such as antioxidants and chelate-formers. As antioxidants there are used, for example, vitamin E, butylated hydroxytoluene, butylated hydroxyanisole, ascorbic acid, ascorbyl palmitate and/or sodium hydrogen sulfite. As chelate-formers there are used, for example, disodium ethylenediaminetetraacetic acid, potassium citrate, sodium citrate and/or citric acid. For stabilising simvastatin, butylated hydroxyanisole and ascorbic acid together with citric acid are preferably used; cf. “Rote Liste”.

The statin(s) may be granulated or pelletised together with the stabiliser(s) and, optionally, excipients in accordance with known methods. The statin/stabiliser particles may accordingly be present in the form of granules of pellets.

The statin/stabiliser particles have a diameter of, for example, from 44 μm to 4 mm (325 to 5 U.S. mesh), preferably from 300 to 1000 μm.

The statin/stabiliser particles may be provided with a protective encapsulation of one or more polymers.

The layer thickness of the protective encapsulation is, for example, from a few μm to 90 μm, preferably about 20 μm.

As protective encapsulation there is suitable any physiologically acceptable polymer, for example metodextrin, gum arabic, waxes, gelatin, polyvinylpyrrolidone, acrylates, methacrylates or polyethylene glycols and also cellulose derivatives, e.g. hydroxypropyl methylcellulose, hydroxypropyl cellulose, cellulose acetate, ethyl cellulose and/or shellac or also sucrose. Cellulose derivatives are preferably used. Mixtures of a plurality of the mentioned substances may also be used.

The encapsulated statin/stabiliser particles may be present in the form of granules or pellets, preferably granules.

The encapsulated statin/stabiliser particles may have a diameter of from 0.1 to 2 mm, preferably from 0.15 to 0.5 mm.

A protective encapsulation for the statin/stabiliser particles may be produced using the following methods: fluidised-bed coating, pan coating, wet granulation, atomising granulation, build-up granulation or compacting.

Fluidised-bed coating is carried out, for example, using the top spray method, Wuster bottom spray method or tangential spray method (cf. also “Air suspension coating for multiparticulates”, D. Jones, Drug Development and Industrial Pharmacy, 20(20), 1994, p. 3175-3206). In those procedures, the statin/stabiliser particles are kept in a suspended state by a stream of air and sprayed with the polymer(s). The polymer(s) and, optionally, excipients (see below) may be present in solution with organic solvents and/or water, suspension, emulsion, latex or in the melted state.

In the pan coating method, a solution of the polymer(s) is sprayed into a rotating pan containing statin/stabiliser particles.

Wet granulation comprises mixing the ingredients such as polymer(s), statin/stabiliser particles, optionally excipients, and solvent (preferably water) using a suitable mixer or kneader (e.g. having a planetary, ploughshare or intensive mixer), wet-screening, drying (e.g. in a fluidised-bed, circulating-air or vacuum dryer) and dry-screening.

For atomising granulation, the statin/stabiliser particles are first dispersed in a solution of the polymer(s). The statin/polymer suspension is then atomised in a drying or cooling tower. The resulting droplets dry or solidify in a drying or cooling gas to form granules. A variant comprises droplet formation wherein a laminar thread of liquid flows out of an orifice and is constricted by regular interferences. As a result of the surface tension, spherical droplets are formed.

In build-up granulation, a solution or suspension of statin(s), stabiliser(s), binders and/or excipients is sprayed onto an inert core, for example of sugar or cellulose. The resulting active ingredient layer is then covered with a protective polymer layer. Crystalline, stabilised statin may also be used as the core. Build-up granulation may be performed using a tangential granulator.

In dry granulation (compacting), the statin/stabiliser particles are granulated under high pressure together with the polymer(s) and, optionally, excipients in powder form without the addition of liquid.

The statin(s) may also be stabilised and encapsulated at the same time by means of extrusion. In that process, the statin(s) is/are mixed with the polymer(s), stabiliser(s) and, optionally, excipients, a granulating liquid is added, homogenisation is carried out to form a mass having a specific plasticity and, finally, extrusion is carried out to form pellets.

The active ingredient combination according to the invention may comprise one or more ACE inhibitors, such as captopril, ceranapril, zofenopril, lisinopril, enalapril, quinapril, imidapril, fosinopril, moexipril, perindopril, spirapril, cilazapril, trandolapril and/or ramipril. Ramipril is preferably used.

The ACE inhibitors may be used in the form of pharmaceutically safe salts, hydrates, solvates and also in the form of derivatives. ACE inhibitors in the form of an ester, especially a C1-C4alkyl ester are used as prodrugs which are then converted into the active metabolites (=carboxylic acids) in the body. For example, both ramipril, an ester prodrug, and also its active metabolite, the dicarboxylic acid ramiprilate, may be used as ACE inhibitors.

As salts of the ACE inhibitors there are suitable, for example, acid addition salts with organic or inorganic acids. Suitable organic carboxylic acids include salicylic acid, maleic acid, tartaric acid, citric acid, adipic acid, sorbic acid, malonic acid, 1,4-butanedioic acid, malic acid, pivalic acid, succinic acid, nicotinic acid, isonicotinic acid, furan-2-carboxylic acid, acetic acid, benzoic acid, fatty acids such as, for example, lauric acid, myristic acid or oleic acid, and suitable inorganic acids include, for example, hydrochloric acid, hydrobromic acid, hydriodic acid, nitric acid, sulfuric acid and/or phosphoric acid.

The ACE inhibitors have a tendency to decomposition reactions such as hydrolysis, cyclisation or oxidation (cf., for example, EP 0 280 999 B1), which are accelerated by acids or bases. They may be stabilised using buffer substances such as sodium dihydrogen phosphate, sodium citrate, sodium carbonate, sodium hydrogen carbonate or tris(hydroxymethyl)aminomethane; cf., for example, EP 0317 878 B1.

The unit dose of the combination according to the invention may comprise, for example: from 0.1 to 100 mg, preferably from 10 to 80 mg, especially from 10 to 40 mg, of statin and from 0.1 to 500 mg, preferably from 1 to 50 mg, especially from 1 to 15 mg, of ACE inhibitor.

Tablets or capsules are suitable as the pharmaceutical formulation. Those presentation forms may comprise carrier materials, fillers, binders, lubricants, preservatives, stabilisers, bulking agent (e.g. mannitol), aromas or coloured pigments.

The following excipients may be used in granule and tablet production: fillers such as cellulose and cellulose derivatives (e.g. microcrystalline cellulose), sugars (e.g. lactose, fructose or dextrose), sugar alcohols (e.g. mannitol or sorbitol), starch, inorganic fillers such as calcium phosphate, calcium sulfate, sodium calcium phosphate, sodium chloride or kaolin, binders such as gelatin, starch or cellulose derivatives (e.g. hydroxypropyl cellulose, hydroxypropyl methylcellulose, methyl cellulose or sodium carboxymethyl cellulose), polyvinylpyrrolidone, sugars (e.g. sucrose, glucose or dextrose), natural gum (e.g. sodium alginate, gum arabic or pectin), disintegrants such as starch and starch derivatives (e.g. sodium carboxylmethyl starch), cross-linked polyvinylpyrrolidone, unmodified or modified cellulose (e.g. sodium carboxymethyl cellulose or cross-linked carboxymethyl cellulose), alginates, flow agents such as microcrystalline cellulose, lubricants such as magnesium stearate, calcium stearate, stearic acid, sodium stearyl fumarate, polyethylene glycol or palmitic acid, sweeteners such as saccharin or aspartame, flavourings such as orange, peppermint or cherry.

For capsules, additional excipients may be used: solvents (e.g. water, alcohol, natural or synthetic oils), solubilisation agents (e.g. glycerol or glycol derivatives), wetting agents (e.g. polysorbate or sodium lauryl sulfate), viscosity-increasing agents and/or pH-adjusters. The main constituents of the shells of capsule formulations are, for example, hydroxypropyl methylcellulose or gelatin.

The tablets or capsules may have a coating, for example of shellac, sugar or both.

ACE inhibitors and non-encapsulated statin may be processed to form a three-layer tablet. The statin layer is separated from the ACE inhibitor layer by a membrane.

As membrane materials there are suitable excipients having a neutral pH, such as microcrystalline cellulose, pregelatinised starch, calcium phosphate and/or combinations thereof.

The encapsulated statin may be mixed with the ACE inhibitor and, in accordance with standard pharmaceutical procedures, compressed into tablets.

The encapsulated statin in the form of granules and pellets together with the ACE inhibitor in the form of granules or pellets may furthermore be filled into capsules in accordance with standard pharmaceutical procedures.

A mixed tablet of simvastatin and ramipril exhibits faster release of ramipril than a tablet that comprises only the ACE inhibitor as active ingredient.

The invention is illustrated further by the following Examples, but without the scope of the invention being limited thereby.

### EXAMPLE 1

Simvastatin layer

| | |
|---|---|
| Simvastatin | 500.0 mg |
| Microcrystalline cellulose | 500.0 mg |
| Ethanol | 350.0 mg |
| Butylated hydroxyanisole | 1.0 mg |
| Purified water | 1250 mg |
| Ascorbic acid | 125.0 mg |
| Citric acid | 62.5 mg |
| Magnesium stearate | 50.0 mg |
| Pregelatinised starch | 80.0 mg |
| Lactose hydrate | 381.0 mg |

Microcrystalline cellulose and simvastatin are homogenised in a high-shear mixer (Ultraturax). A solution of butylated hydroxyanisole is combined with a solution of ascorbic acid and citric acid in water. The resulting solution together with the microcrystalline cellulose and simvastatin are subjected to wet granulation in a high-shear mixer. Fluidised-bed drying is then carried out at 80° C. and <35% RH. The granules thereby obtained are screened through a 1.0 to 1.5-mm sieve and granulated with magnesium stearate in a cone mixer. The resulting granules are processed together with lactose hydrate, pregelatinised starch and magnesium stearate to form a simvastatin compression mass.

Ramipril Layer

| | |
|---|---|
| Ramipril | 10.0 mg |
| Sodium hydrogen carbonate | 8.0 mg |
| Hydroxypropyl methylcellulose | 30.0 mg |
| Microcrystalline cellulose | 96.8 mg |
| Pregelatinised starch | 94.6 mg |
| Sodium stearyl fumarate | 0.6 mg |

Ramipril and sodium hydrogen carbonate are wet-granulated with water. The granules are processed with Methocel, microcrystalline cellulose, starch and sodium stearyl fumarate to form a ramipril compression mass.

Three-Layer Tablet

The simvastatin layer (1700 mg/tablet) is processed together with a layer of microcrystalline cellulose (240 mg/tablet) and the ramipril layer (240 mg/tablet) on a rotary tablet press to form a three-layer tablet.

### EXAMPLE 2

Simvastatin Layer

| | |
|---|---|
| Simvastatin | 40.0 mg |
| Microcrystalline cellulose | 20.0 mg |
| Butylated hydroxyanisole | 0.08 mg |
| Ascorbic acid | 10.0 mg |
| Citric acid monohydrate | 5.0 mg |
| Magnesium stearate | 4.0 mg |
| Pregelatinised starch | 40.0 mg |
| Lactose monohydrate | 281.0 mg |

Preparation of the simvastatin compression mass is carried out as in Example 1.

Ramipril Layer

| | |
|---|---|
| Ramipril | 10.0 mg |
| Sodium hydrogen carbonate | 10.0 mg |
| Hydroxypropyl methylcellulose | 25.0 mg |
| Microcrystalline cellulose | 97.0 mg |
| Pregelatinised starch | 96.5 mg |
| Sodium stearyl fumarate | 1.5 mg |

Processing to produce a ramipril compression mass is carried out analogously to Example 1.

Three-Layer Tablet

The simvastatin layer (400 mg/tablet) is processed together with a layer of microcrystalline cellulose (240 mg/tablet) and the ramipril layer (240 mg/tablet) on a rotary tablet press to form a three-layer tablet.

### EXAMPLE 3

Simvastatin Layer

| | |
|---|---|
| Simvastatin | 40.0 mg |
| Microcrystalline cellulose | 40.0 mg |
| Butylated hydroxyanisole | 0.08 mg |
| Ascorbic acid | 10.0 mg |
| Citric acid | 5.0 mg |
| Magnesium stearate | 4.0 mg |
| Pregelatinised starch | 80.0 mg |
| Lactose monohydrate | 381.0 mg |

Preparation of the simvastatin compression mass is carried out as in Example 1.

Ramipril Layer

| | |
|---|---|
| Ramipril | 10.0 mg |
| Sodium hydrogen carbonate | 7.0 mg |
| Hydroxypropyl methylcellulose | 40.0 mg |
| Microcrystalline cellulose | 91.0 mg |
| Pregelatinised starch | 91.0 mg |
| Magnesium stearate | 1.0 mg |

Processing to produce a ramipril compression mass is carried out analogously to Example 1.

Three-Layer Tablet

The simvastatin layer (560 mg/tablet) is processed together with a layer of microcrystalline cellulose (240 mg/tablet) and the ramipril layer (240 mg/tablet) on a rotary tablet press to form a three-layer tablet.

### EXAMPLE 4

Simvastatin Layer

| | |
|---|---|
| Simvastatin | 40.0 mg |
| Microcrystalline cellulose | 20.0 mg |
| Butylated hydroxyanisole | 0.08 mg |
| Ascorbic acid | 10.0 mg |
| Citric acid monohydrate | 5.0 mg |
| Magnesium stearate | 4.0 mg |
| Pregelatinised starch | 40.0 mg |
| Lactose monohydrate | 281.0 mg |

Preparation of the simvastatin compression mass is carried out as in Example 1.

Ramipril Layer

| | |
|---|---|
| Ramipril | 10.0 mg |
| Sodium hydrogen carbonate | 10.0 mg |
| Hydroxypropyl methylcellulose | 35.0 mg |
| Microcrystalline cellulose | 91.5 mg |
| Pregelatinised starch | 91.5 mg |
| Magnesium stearate | 2.0 mg |

Processing to produce a ramipril compression mass is carried out analogously to Example 1.

Three-Layer Tablet

The simvastatin layer (400 mg/tablet) is processed together with a layer of microcrystalline cellulose (240 mg/tablet) and the ramipril layer (240 mg/tablet) on a rotary tablet press to form a three-layer tablet.

### EXAMPLE 5

Simvastatin Layer

| | |
|---|---|
| Simvastatin | 40.0 mg |
| Microcrystalline cellulose | 40.0 mg |
| Butylated hydroxyanisole | 0.08 mg |
| Ascorbic acid | 10.0 mg |
| Citric acid | 5.0 mg |
| Magnesium stearate | 4.0 mg |
| Pregelatinised starch | 80.0 mg |
| Lactose monohydrate | 381.0 mg |

Preparation of the simvastatin compression mass is carded out as in Example 1.

Ramipril Layer

| | |
|---|---|
| Ramipril | 10.0 mg |
| Sodium hydrogen carbonate | 45.0 mg |
| Hydroxypropyl methylcellulose | 35.0 mg |
| Microcrystalline cellulose | 86.6 mg |
| Pregelatinised starch | 81.0 mg |
| Magnesium stearate | 1.2 mg |
| Stearic acid | 1.2 mg |

Processing to produce a ramipril compression mass is carried out analogously to Example 1.

Three-Layer Tablet

The simvastatin layer (560 mg/tablet) is processed together with a layer of microcrystalline cellulose (240 mg/tablet) and the ramipril layer (260 mg/tablet) on a rotary tablet press to form a three-layer tablet.

1. Pharmaceutical formulation having a content of at least one statin and at least one ACE inhibitor, wherein the at least one statin and the at least one ACE inhibitor are separated by a physiologically acceptable inert material.

2. Pharmaceutical formulation according to claim 1, wherein the at least one statin is present with at least one statin stabiliser and, optionally, the at least one ACE inhibitor is present with at least one ACE inhibitor stabiliser.

3. Pharmaceutical formulation according to claim 1 and/or claim 2, having a buffer substance, antioxidant and/or chelate-former as statin stabiliser.

4. Pharmaceutical formulation according to claim 3, having vitamin E, butylated hydroxytoluene, butylated hydroxyanisole, ascorbic acid, ascorbyl palmitate and/or sodium hydrogen sulfite as antioxidant.

5. Pharmaceutical formulation according to claim 3 and/or 4, having disodium ethylenediaminetetraacetic acid, potassium citrate, sodium citrate and/or citric acid as chelate-former.

6. Pharmaceutical formulation according to at least one of the preceding claims, having a buffer substance as ACE inhibitor stabiliser.

7. Pharmaceutical formulation according to at least one of claims 3 to 6, having sodium dihydrogen phosphate, sodium citrate, sodium carbonate, sodium hydrogen carbonate and/or tris(hydroxymethyl)aminomethane as buffer substance.

8. Pharmaceutical formulation according to at least one of the preceding claims, having a natural polymer, a synthetic polymer, a non-simple sugar and/or an inorganic material as inert material.

9. Pharmaceutical formulation according to at least one of the preceding claims, wherein the separating physiologically acceptable material is provided in the form of an encapsulation of the combination of at least one statin and at least one statin stabiliser.

10. Pharmaceutical formulation according to at least one of the preceding claims and especially claim 9, wherein the at least one statin is present in admixture with at least one statin stabiliser in the form of particles, especially granules or pellets, the particles being encapsulated by the separating physiologically acceptable material.

11. Pharmaceutical formulation according to claim 10, wherein the formulation is obtainable by encapsulating particles of a mixture of at least one statin and at least one statin stabiliser by the separating physiologically acceptable material.

12. Pharmaceutical formulation according to at least one of the preceding claims and especially claim 10, wherein the formulation is obtainable by embedding, and thereby encapsulating, the at least one statin and the at least one statin stabiliser in the separating physiologically acceptable material.

13. Pharmaceutical formulation according to at least one of the preceding claims, wherein the at least one statin and the at least one statin stabiliser are encapsulated by sucrose, metodextrin, gum arabic, wax, polyvinylpyrrolidone, gelatin, cellulose, a cellulose derivative and/or shellac as inert material.

14. Pharmaceutical formulation according to claim 13, having hydroxypropyl methylcellulose, hydroxypropyl cellulose, cellulose acetate and/or ethyl cellulose as cellulose derivative.

15. Pharmaceutical formulation according to at least one of claims 8 to 14, wherein the at least one statin and the at least one statin stabiliser are encapsulated by a polyacrylate, polymethacrylate, polyether and/or polyether alcohol, especially polyethylene glycol, as polymer.

16. Pharmaceutical formulation according to at least one of the preceding claims, wherein the formulation has been filled into capsules, especially a formulation comprising encapsulated statin in the form of granules or pellets and ACE inhibitor in the form of granules or pellets.

17. Pharmaceutical formulation according to at least one of claims 1 to 15, wherein the formulation has been further processed into tablets.

18. Pharmaceutical formulation according to at least one of claims 1 to 8, wherein the formulation is provided in the form of tablets in which the at least one statin and the at least one ACE inhibitor are separated by a layer or a membrane of a physiologically acceptable inert material of neutral pH.

19. Pharmaceutical formulation according to claim 18, having calcium phosphate as inorganic inert material.

20. Pharmaceutical formulation according to claim 18, having microcrystalline cellulose and/or pregelatinised starch as inert material.

21. Pharmaceutical formulation according to at least one of the preceding claims, having atorvastatin, rosuvastatin, fluvastatin, pravastatin, cerivastatin and/or simvastatin as statin.

22. Pharmaceutical formulation according to at least one of the preceding claims and especially according to claim 21, having at least one statin in each case in the form of the carboxylic acid, a carboxylic acid salt, a lactone, an ester, a hydrate, an alcoholate and/or a tautomer.

23. Pharmaceutical formulation according to at least one of the preceding claims, having captopril, ceranapril, zofenopril, lisinopril, enalapril, quinapril, imidapril, fosinopril, moexipril, perindopril, spirapril, cilazapril, trandolapril and/or ramipril as ACE inhibitor.

24. Pharmaceutical formulation according to at least one of the preceding claims and especially according to claim 23, having at least one ACE inhibitor in each case in the form of the carboxylic acid, an ester, a salt, an acid addition salt, a hydrate and/or a solvate as ACE inhibitor.
